(19) United States
(12) Patent Application Publication
(10) Pub. No.: US 2010/0049328 A1
(43) Pub. Date: Feb. 25, 2010

(54) GASTROENTEROLOGICAL MEDICAL DEVICE, IN PARTICULAR STENT FOR THE GALL OR PANCREATIC DUCT

(76) Inventor: Peter HILDEBRANDT, Nurnberg (DE)

Correspondence Address: BROWDY AND NEIMARK, P.L.L.C.;624 NINTH STREET, NW, SUITE 300, WASHINGTON, DC, 20001-5303, US

(21) Appl. No.: 12/195,266

(22) Filed: Aug. 20, 2008

(51) Int. Cl. A61F 2/82

(52) U.S. Cl. 623/23.7

(57) ABSTRACT

A gastroenterological medical device, particularly a stent for the gall or pancreatic duct, has a substantially tubular, intrinsically stable carrier that is provided with a spacer layer that is attached to the carrier surface, and a layer of an electronegative glycosaminoglycan that is attached to the spacer layer.

### BACKGROUND OF THE INVENTION

1. Field of the Invention

The invention relates to a gastroenterological medical device comprising a substantially tubular, intrinsically stable carrier.

2. Background Art

Medical devices of this type are usually referred to in the professional jargon as stents, prostheses or catheters. Gastroenterological stents and prostheses as a rule are used for dilating and maintaining ducts and pathways in the body that are pathologically closed or narrowed in diameter. In terms of catheters, nasal and percutaneous feeding tubes may be mentioned as example applications for the specified gastroenterological medical devices.

For reasons of simplicity, in the description of the prior art and of the invention that follows, the previously mentioned stents will be discussed in more detail as examples:

Gastroenterological stents are indispensable implants for patients with gall and pancreatic duct disorders. A large selection of stent designs and materials are already available today. The design and material, as a rule, depend on the site where the stent will be placed and on the implantation technique that will be used. Tubular carriers that are made from an expansible lattice material are common, for example. Tubing-like stents with closed walls made from a polymer material are often used as well. The stent is inserted endoscopically into the gall or pancreatic duct. It remains at the implantation site and serves as a shoring to prevent a reoccurrence of the narrowing.

A serious problem resides in that in all materials for gastroenterological medical devices that have been used and tested so far, a bacterial film and deposits—so-called biofilm—occur on the surface of the medical device i after only several days of contact with bile, pancreas secretion, stomach or intestinal contents. This leads to severe dysfunctions, such as infections and a partial or complete blockage of the tubular medical device, thereby jeopardizing the long-term medical success. Numerous studies with regard to this problem have dealt with various medical materials. However, so far no real durable resistance to the above-mentioned effects has been found in any material.

One approach from the prior art to solving the biofilm problem can be found in U.S. Pat. No. 6,228,393 B1. This document discloses a coating that releases active substances, such as antibiotics, for instance. This is intended to prevent the formation of a bacteria film on the surface. The prior art discloses numerous additional patents that are based on the principle of the release of an active substance. This requires a reservoir of active substances, however, which is inevitably limited. Once this reservoir is depleted, the surface returns to its original properties in terms of its susceptibility to bacterial colonization. This makes the application appear of little benefit in cases of extended indwelling times.

Another example of corresponding prior art measures can be found in U.S. Pat No. 6,361,567 B1. This document discloses an antimicrobial coating for use in medical implants. The principle of action of the coating is based on the use of antimicrobial metal ions, such as silver, gold, or a combination of both. The prior art discloses numerous additional patents that are based on the principle of the antimicrobial properties of metal ions. As has, however, been found, the inhibition of bacterial colonization that is achievable by this prior art is still in need of improvement.

EP 0 890 367 B1 discloses a glycosaminoglycan coating on urological implants in contact with urine. In this environment, the glycosamino-glycans act as inhibitors, i.e. by adsorption to molecules and crystal surfaces in the urine, thereby blocking their binding sites. This prevents the growth of large crystal structures. The action of an inhibitor in this context is usually limited to one or a few particle types. For example, nephrocalcin binds to calcium ions and heparin binds to oxalate ions and oxalate crystallites as inhibitors. A covalent attachment of inhibitors to the substrate consequently creates a layer to which urine components are attached in such a manner that no additional components can deposit and crystal growth is blocked. This mechanism has absolutely no relevance for the use of a gastroenterological implant, in particular for the gall and pancreatic duct.

### SUMMARY OF THE INVENTION

The invention is now based on the object of providing an implant that is optimized for gastroenterological purposes, in which bacterial colonization and biofilm formation are effectively inhibited.

The invention accordingly proposes a gastroenterological medical device with a substantially tubular, intrinsically stable carrier in which the surface of the carrier has a spacer molecule layer attached to it, on which, in turn, a layer of an electronegative glycosaminoglycan is attached.

The present invention, in contrast to the described crystallization-inhibiting mechanisms in urinary stents, is based on bio-medical processes for inhibiting the bacterial colonization and biofilm formation in contact with bile, pancreas secretion, stomach or intestinal content:

Tire formation of biofilm in the normal case occurs in the following steps: the first step consists of microorganisms depositing on the implant surface, followed by their sturdy anchoring by means of microbial adhesion and exopolymer production. After that, a strong growth of the microorganisms can usually be observed.

The biofilm, in most cases, is composed of three layers: the conditioning film, ensures the anchoring to the implant surface, the intermediate biofilm layer consists of microorganisms, and the surface film represents the outer surface of the biofilm, on which planktonic organisms can be exchanged with the environment.

Glycosaminoglycans are substances that naturally occur dissolved in body fluids on one hand, and a component of the uppermost cell layers of vessels, pathways and ducts in the body, on the other hand. They possess a strong electronegativity and thus repel microorganisms. This, surprisingly, also makes glycosaminoglycans excellent for the coating of implants for which protection against the formation of biofilm in the contact with bile, pancreas secretion, stomach or intestinal contents is desired. This mechanism is totally different to the one in urine. In urine the coating blocks crystal growth no matter whether microorganisms are present or not. In many cases urine is sterile and crystallisation occurs still on implant surfaces in the absence of microorganisms.

As has been found with regard to the invention, a direct attachment of glycosaminoglycans to the carrier surface is not sufficient in this case. The reason being that die direct attachment results in a weakening of the electronegative field, which strongly weakens the effectiveness. The insertion of the claimed spacer between the carrier surface and the glycosaminoglycan layer increases the space between them and thus is able to prevent the above-explained weakening of the electronegative field. Furthermore, this method of attachment makes the long-term stability of the coating possible, and therefore also that of its action.

In accordance with preferred embodiments, the carrier may consist of a polymer, such as silicone, polyurethane, polyethylene, or the like, on one hand; or of metals or metal alloys, such as medical-grade stainless steel, titanium, nickel-titanium, or the like, on the other hand, in each case with an amorphous silicon carbide coating as an active substrate surface. A configuration of a metal base structure with a polymer coating is feasible as well. Also, the selection of the chemical components of the spacer layer is made in dependence upon the material properties of the carrier and its substrate surface.

Preferably, a heparin layer that is immobilized on the spacer layer is used as the glycosaminoglycan layer. The heparin layer is attached to and immobilized on the substrate surface by means of a covalent attachment.

In summary, experimental studies with gastroenterological stents equipped in accordance with the invention, in which a polyethylene carrier was used, revealed a strong suppression of the bacterial colonization and biofilm formation in contact with gall and pancreas secretion. The experimental studies also demonstrated a sufficiently long-term stability of the heparin bond on the spacer layer.

Additional features, details, and advantages of the object of the invention will become apparent from the description below, in which an exemplary embodiment of the object of the invention is explained in more detail in conjunction with the appended drawing.

### BRIEF DESCRIPTION OF THE DRAWING

FIG. 1 is a schematic, sectional perspective view of a gastroenterological stent;

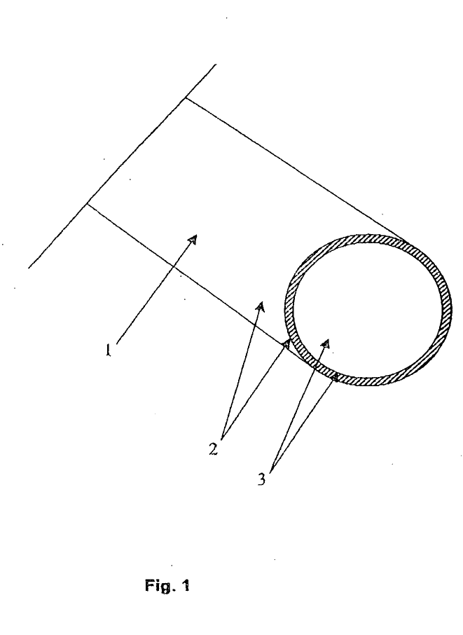

FIG. 2 is a cross-sectional view of a delivery system with the gastroenterological stent of FIG. 1 loaded therein;

FIG. 3 is a schematic of the delivery system of FIG. 2 emerging from a working channel of an endoscope positioned in close proximity to a biliary tree; and

FIG. 4 is a schematic of the gastroenterological stent deployed from the delivery system of FIG. 2.

### DESCRIPTION OF THE PREFERRED EMBODIMENT

The shown stent has a carrier 1, consisting, for example, of polyethylene. On its outer surface 2 and inner surface 3 a layer that is not shown in detail of a glycosaminoglycan is covalently attached via a spacer.

Described below in me form of a list of individual process steps is an exemplary coating process for the immobilization of heparin on a sample in the form of a polyethylene surface of the stent carrier:
Place sample in a solution of 2 g potassium permanganate, 0.5 l sulfuric acid and 0.5 l deionised water for 2 minutes.
Rinse sample in 1 l deionised water.
Repeat rinsing.
Place sample in a solution of 200 μl polyethylene imine and 1 l deionised water for 5 minutes for formation of the spacer layer.
Rinse sample in 1 l deionised water.
Place sample in a solution of 10 mg cyanoboro hybride, 5 mg heparin and 1 l deionised water for 2 hours for formation of the glycosaminoglycan layer on the spacer layer.
Rinse sample in 1 l deionised water.

For the spacer layer, various alternatives are feasible. For example, the spacer layer may be formed on the basis of a propylsiloxyl compound, such as, e.g. a partially substituted 3-(adipinic-acid-amino)propylsiloxyl compound. Provision may be made also for the use of a photoactive benzophenone compound, such as e.g. a Fmoc-p-Bz-Phe-OH solution in N,N′-dimethyl formamide as a photoactive benzophenone compound. The glycosaminoglycan layer may also be formed of a layer of a synthetic heparin derivative—optionally in combination of various glycosaminoglycans—that is immobilized on the spacer layer.

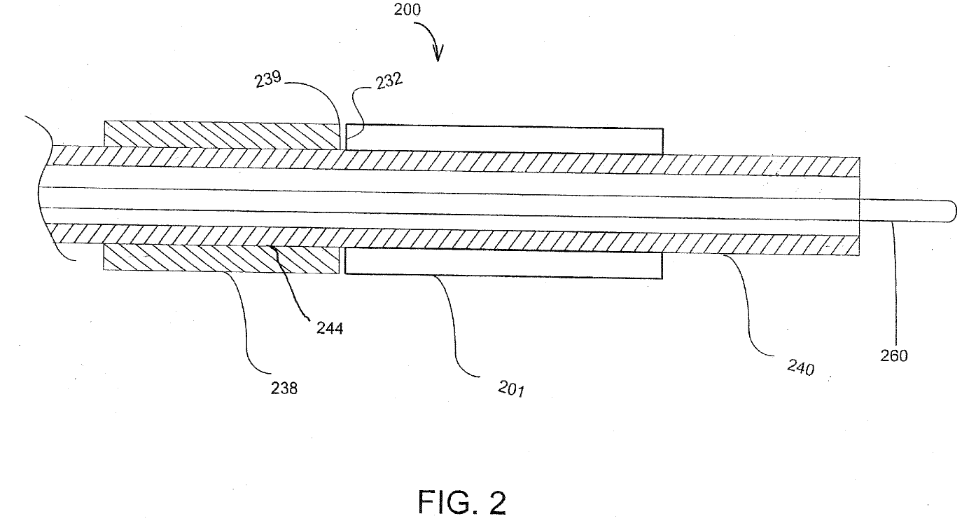

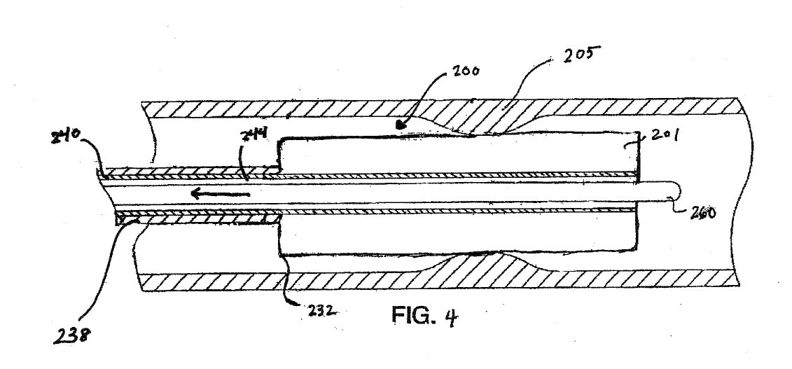

A method of delivering the above-described gastroenterological device to a target gastrointestinal site 205 (FIGS. 2 and 4) of a patient will now be described. The terms “proximal” and “distal” as used herein are intended—to have a reference point relative to the user. Specifically, throughout the specification, the terms “distal” and “distally” shall denote a position, direction, or orientation that is generally away from the user, and the terms “proximal” and “proximally” shall denote a position, direction, or orientation that is generally towards the user.

FIG. 2 illustrates one exemplary delivery system 200 that may be used to deliver the stent carrier 201. The delivery system 200 is shown to include an outer pushing catheter 238 and an inner guiding catheter 240. The outer pushing catheter 238 is coaxially disposed over the inner guiding catheter 240. The stent carrier 201 is shown in a loaded configuration over a distal portion of the inner guiding catheter 240. The stent carrier 201 in its loaded configuration has a proximal end 232 which abuts against the distal end 239 of the outer pushing catheter 238.

The stent carrier 201 comprises a spacer layer attached to the outer surface of the stent carrier 201 and a glycosaminoglycan layer covalently attached to the spacer film, preferably in accordance with the procedure described above. In a preferred embodiment, the glycosaminoglycan layer is formed from fondaparinux sodium, the spacer layer is formed from polyethyleneimine, and the stent carrier 201 is formed from polyethylene.

The glycosaminoglycan layer is preferably contained in an effective amount to inhibit biofilm formation along the stent 201 after the stent has been deployed to the target site 205 in the gastrointestinal tract.

Deployment of stent carrier 201 using the delivery system 200 with stent carrier 201 loaded therein may be achieved in several ways as known in the art, including several non-endoscopic procedures, such as, for example, percutaneous advancement of the delivery system 200. Preferably, the delivery system 200 is delivered and deployed into a target gastrointestinal site 205 using an endoscopic procedure, as will now be described.

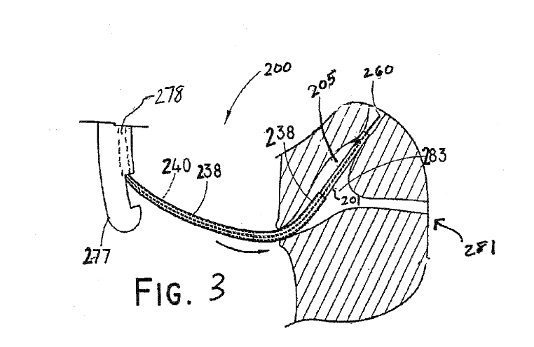

After loading the stent carrier 201 at the distal portion of the delivery system 200 coaxially disposed over the inner guiding catheter 240, the delivery system 200 may be navigated to the target gastrointestinal site 205. Preferably, cannulation of the target gastrointestinal site 205 is initially achieved by maneuvering a wire guide 260 therethrough, as shown in FIG. 4. The delivery system 200 is fed over the wire guide 260 and inserted into a working channel 278 of an endoscope 277 at a proximal end of the channel 278. As the delivery system 200 continues to be advanced through the working channel 278 over wire guide 260, the distal portion of delivery system 200 emerges from the working channel 278, as shown by the arrow in FIG. 3. Thereafter, the distal portion of the delivery system 200 is maneuvered into the desired duct 283 of the biliary tree 281. The delivery system 200 is preferably positioned so as to have a distal end of the stent carrier 201 extending past the target site 205 (FIG. 4) and the proximal end 232 of the stent 201 disposed within a duodenum of the patient.

Having maneuvered the delivery system 200 to the desired target gastrointestinal site 205, deployment of stent carrier 201 may occur. The outer pushing catheter 238 is preferably maintained in position while the inner guiding catheter 240 is proximally withdrawn, as shown by the arrow in. FIG. 4, from tire lumen 244 of outer pushing catheter 238. Inner guiding catheter 240 continues to be proximally retracted relative to outer pushing catheter 238 until stent carrier 201 is completely disengaged from the inner guiding catheter 240. In particular, the proximal end 232 of the stent carrier 201 is disposed beyond the distal ends 239 of outer pushing catheter 238 and inner guiding catheter 240, as shown in FIG. 4, so as to achieve deployment of the stent carrier 201 at the target gastrointestinal site 205.

After the stent carrier 201 is in the desired deployed position, the delivery system 200 may be withdrawn.. Wire guide 260 is preferably maintained in position within the desired duct 283 of the biliary tree 281. If desired, subsequent deployment of additional stent carriers 201 having a spacer film and a glycosaminoglycan covalently attached to the spacer film may be delivered and deployed over wire guide 260 using the above-described procedure.

Although the above-described delivery technique is described with the delivery system 200 being introduced completely over the proximal end of the wire guide 260, the wire guide 260 and delivery system 200 may be coupled for only a portion of their length in a short-wire exchange. The short-wire exchange allows a much shorter wire guide to be used since a much shorter length of wire guide is needed to extend outside a patient to permit the practitioner to maintain control of the wire guide during the exchange.

The glycosaminoglycan may sufficiently inhibit biofilm formation on a surface of the stent carrier 201 which is now deployed at target gastrointestinal site 205. Such inhibition of biofilm formation may help to substantially inhibit occlusion of the gastroenterological medical device, thereby increasing the duration of patency of the stent 201 as compared to stents not having the above-described spacer layer and covalently attached glycosaminoglycan layer.

### COMPARATIVE EXAMPLE

An independent study was conducted to evaluate the efficacy of heparin coated biliary stents to substantially reduce stent occlusion as compared to standard uncoated stents. All the stents used had a diameter of 10Fr and were formed from polyethylene. The coated stents included a spacer layer and a heparin layer. The spacer layer was added to the surface of the stents in accordance with the procedure described above. The heparin layer was covalently attached to the spacer layer also as described above. Before implantation, the weight of all stents was determined. Due to randomisation, coated or standard stents were then implanted in jaundiced patients suffering from malignant biliary obstruction. Scheduled stent exchange was performed after 90 days implanting new coated or standard stents according to the cross over design for the same duration. The implanted stents were retrieved after 90 days and thereafter stored at—18° C. Immediately before analysis, the stents were dried at 50° C. for 24 hours, then weighed and finally longitudinally opened to visualize incrustation. Occlusion was measured by the increase of stent dry weight. Statistical analysis was performed using the Wilcoxon-Test.

In total 32 patients were randomized. Twenty-two patients dropped out due to short duration of stent implantation or missing cross over. In 10 patients (3 male/7 female, 58-79 yrs.) study was completed. Premature stent removal was necessary in 3/10 standard stents, because of new jaundice or cholangitis, but in none of the coated stents. After longitudinal incision, all three stents showed total or partial occlusion. Altogether, coated stents showed less visible occlusion and discolouration than standard stents. On average the weight of standard stents was twice as high as of coated stents (standard: 32±12 (16-56) mg; coated: 15±4 (9-24) mg), although the duration of stent implantation was not significantly different between both groups (standard: 80±21 (30-106) days; coated: 87±13 (56-101) days). In total, the weight of removed coated stents were lower than the weight of the standard stents in 9/10 patients, thereby allowing the coated stents to remain in situ for a longer period of time to reduce the frequency of scheduled stent exchange.

1. A gastroenterological medical device, in particular a stent for the gall or pancreatic duct, comprising a substantially tubular, intrinsically stable carrier (1), adapted for insertion into the gastrointestinal tract to provide a wall support for the gastrointestinal tract, a spacer layer attached to the carrier surface (2, 3) and means for inhibiting biofilm formation from digestive juices on said carrier and said spacer layer, said means comprising an electronegative glycosaminoglycan attached to the spacer layer, wherein by the spacer layer the glycosaminoglycan is bonded in an undeformed condition.

2. A gastroenterological medical device according to claim 1, wherein the carrier (1) consists of a polymer, in particular silicone, polyurethane, polyethylene, polyvinyl chloride, or the like.

3. A gastroenterological medical device according to claim 1, wherein the carrier (1) consists of an intrinsically stable base structure, in particular of metal, with a coating consisting of a polymer, in particular silicone, polyurethane, polyvinyl chloride, or the like.

4. A gastroenterological medical device according to claim 1, wherein the carrier (1) consists of tantalum, a titanium alloy, medical-grade stainless steel, or pyrolytic carbon, in each case with an amorphous silicon carbide coating as an activated substrate surface.

5. A gastroenterological medical device according to claim 1, wherein the spacer layer is formed on the basis of a polyethylenimine compound.

6. A gastroenterological medical device according to claim 1, wherein the spacer layer is formed on the basis of a propylsiloxyl compound.

7. A gastroenterological medical device according to claim 6, wherein the spacer layer is formed from a partially substituted 3-(adipinic-acid-amino)propylsiloxyl compound.

8. A gastroenterological medical device according to claim 4, wherein the spacer layer is formed on the basis of a photoactive benzophenone compound.

9. A gastroenterological medical device according to claim 8, comprising a Fmoc-p-Bz-Phe-OH solution in N,N′-dimethyl formamide as a photoactive benzophenone compound.

10. A gastroenterological medical device according to claim 1, wherein the glycosaminoglycan layer is formed of a heparin layer that is immobilized on the spacer layer.

11. A gastroenterological medical device according to claim 1, wherein the glycosaminoglycan layer is formed of a layer of a synthetic heparin derivative that is immobilized on the spacer layer.

12. A gastroenterological medical device according to claim 1, wherein the glycosaminoglycan layer is formed of a layer of a combination of different glycosaminoglycans that is immobilized on the spacer layer.

13. A gastroenterological medical device according to claim 1, wherein the carrier surface (2) is activated by sulfuric acid.

14. A gastroenterological medical device according to claim 1, wherein the carrier surface (2) is activated by potassium permanganate.

15. A gastroenterological medical device according to claim 1, wherein the carrier surface (2) is activated by sulfuric acid and potassium permanganate in one step.

16. A gastroenterological medical device according to claim 13, wherein the spacer layer is directly bonded to the activated carrier surface (2).

17. A gastroenterological medical device according to claim 1, wherein the glycosaminoglycan layer is immobilized on the spacer layer using cyanoboro hybride.

18. A method for treating a target gastrointestinal site, comprising the steps of:
providing a gastroenterological medical device comprising a tubular non-expandable stent, a spacer layer attached to an outer surface of the stent, and an electronegative glycosaminoglycan covalently attached in an undeformed condition to the spacer layer; and
disposing the gastroenterological medical device at the target gastrointestinal site.

19. The method of claim 18, wherein the gastrointestinal site comprises a common bile duct within a patient.

20. The method of claim 18, further comprising the steps of:
providing a delivery system comprising an elongate guide member, and an elongate pusher member movably disposed over the elongate guide member, the elongate guide member and the elongate pusher member each having a length sufficient to reach the target gastrointestinal site;
disposing the gastroenterological medical device on a distal portion of the guiding member so as to engage a distal portion of the pusher member;
advancing the delivery system to the target gastrointestinal site; and
deploying the gastroenterological medical device by retracting the guide member proximally relative to the pusher member.

21. The method of claim 20, wherein the elongate guide member comprises a wire guide.

22. The method of claim 20, wherein the elongate guiding member comprises an inner tubular member with a lumen extending at least partially therethrough, and further comprising the steps of:
providing a wire guide, the wire guide being movably disposed through the lumen of the inner tubular member;
advancing the wire guide to the target gastrointestinal site; and
advancing the delivery system along the wire guide and to the target gastrointestinal site.

23. The method of claim 20 wherein the delivery system is advanced to the target gastrointestinal site through a working channel of an endoscope.

24. The method of claim 20 wherein the delivery system is advanced to the target gastro site percutaneously.

25. The method of claim 18, wherein the spacer layer comprises polyethylenenimine.

26. The method of claim 18, wherein the glycosaminoglycan comprises fondaparinux sodium.

27. The method of claim 18, wherein the stent is formed from polyethylene.

28. The method of claim 18, wherein the method includes providing an effective amount of the glycosaminoglycan to sufficiently inhibit formation of a biofilm on an inner surface of the gastroenterological medical device.

29. The method of claim 18, wherein the method includes providing an effective amount of the glycosaminoglycan to sufficiently inhibit occlusion of the gastroenterological medical device by a formation of biofilm on a surface thereof.

30. A gastroenterological medical device according to claim 14, wherein the spacer layer is directly bonded to the activated carrier surface (2).

31. A gastroenterological medical device according to claim 15, wherein the spacer layer is directly bonded to the activated carrier surface (2).
